SEQUENCE LISTING (3 sequences)

SEQ ID NO: 1 - DNA, 21 bp, Homo sapiens
tgcacgtggatcctgagaact

SEQ ID NO: 2 - DNA, 22 bp, Homo sapiens
aattctttgccaaagtgatggg

SEQ ID NO: 3 - DNA, 22 bp, Artificial Sequence
  TaqMan probe
cagcacgttgcccaggagcctg

(19) United States
(12) Patent Application Publication
(10) Pub. No.: US 2008/0287669 A1
(43) Pub. Date: Nov. 20, 2008

(54) METHODS AND COMPOSITIONS FOR IDENTIFYING COMPOUNDS USEFUL IN NUCLEIC ACID PURIFICATION

(76) Inventors: Jeffrey C. BRAMAN, Carlsbad, CA (US); Lee Scott Basehore, Lakeside, CA (US); Natalia Novoradovskaya, San Diego, CA (US)

Correspondence Address: AGILENT TECHOLOGIES INC, P.O BOX 7599, BLDG E , LEGAL, LOVELAND, CO, 80537-0599, US

(21) Appl. No.: 12/120,956

(22) Filed: May 15, 2008

Related U.S. Application Data
(60) Provisional application No. 60/938,264, filed on May 16, 2007.

(51) Int. Cl. C07H 1/00

(52) U.S. Cl. 536/25.41

(57) ABSTRACT

The present invention provides methods, compositions, and kits for identifying compounds useful in nucleic acid purification. The methods of the invention include identifying certain characteristics of organic solvents such as miscibility in water, dielectric constant, and the class of the solvent.

### CROSS-REFERENCE TO RELATED APPLICATIONS

This application relies on and claims the benefit of the filing date of U.S. provisional patent application No. 60/938,264, filed 16 May 2007, the entire disclosure of which is hereby incorporated herein by reference.

### BACKGROUND OF THE INVENTION

1. Field of the Invention

The present invention relates to the field of isolation and purification of biological molecules. More specifically, the present invention pertains to methods, compositions, and kits for identifying substances useful in nucleic acid purification, and to the substances themselves.

2. Description of Related Art

Isolation of biological molecules, such as DNA and RNA, and their subsequent analysis is a fundamental part of molecular biology. Analysis of nucleic acids is crucial to gene expression studies, not just in basic research, but also in the medical field of diagnostic use. For example, diagnostic tools include those for detecting nucleic acid sequences from minute amounts of cells, tissues, and/or biopsy materials, and for detecting viral nucleic acids in blood or plasma. The yield and quality of the nucleic acids isolated and purified from a sample has a critical effect on the success of any subsequent analyses.

Isolation of nucleic acids from a biological sample usually involves lysing the biological sample by, for example, mechanical action and/or chemical action followed by purification of the nucleic acids. Previously, purification of nucleic acids was performed using methods such as cesium chloride density gradient centrifugation (which is time-consuming and expensive) or extraction with phenol (which is considered unhealthy for the user). In a typical final step, ethanol precipitation was used to concentrate the nucleic acids, which resulted in lower yields of the isolated nucleic acids.

Many of the methods currently used to isolate nucleic acids are based on the adsorption of the nucleic acid on glass or silica particles in the presence of a chaotropic salt. In 1933, Alloway reported using absolute alcohol or acetone to precipitate the “active transforming principle” (DNA) from Pneumococcus extracts (Alloway, L., J. Exp. Med. 57: 265-278, 1933). To chemically prove that the material Alloway described was DNA, O. T. Avery, C. M. MacLeod and M. McCarty (Avery, O. T., MacLeod C. M. and McCarty, M., J. Exp. Med. 79: 137-158, 1944) also used ethanol for purifying DNA by precipitation from a saline solution (0.85% NaCl) and spooling the DNA onto a glass rod: “At a critical concentration varying from 0.8 to 1.0 volume of alcohol the active material separates out in the form of fibrous strands that wind themselves around the stirring rod.” Since then, a variety of nucleic acid purification methods have been developed relying on alcohols to precipitate DNA and RNA (Molecular Cloning: A Laboratory Manual, third edition, Sambrook, J. and Russell, D. W., chapters 6 [protocols 6.4 through 6.28] and 7 [protocols 7.4 through 7.18], Cold Spring Harbor Laboratory Press, Cold Spring Harbor, N.Y.).

Vogelstein and Gillespie (Vogelstein, B. and Gillespie, D., PNAS 76: 615-619.) described recovery of DNA, ranging in size from 100 base pairs (bp) to 48,000 bp, from agarose gels by dissolving the agarose in high concentration chaotropic salt followed by acetone precipitation. Alternatively, following chaotropic salt treatment of agarose, DNA was bound to powdered glass (glass fiber filter was also found to bind DNA in the presence of high concentration chaotropic salt). The glass was washed with 50% aqueous buffered ethanol to remove chaotropic salt and DNA was eluted from the glass with low ionic strength buffer, precipitated with ethanol and dissolved in buffer. It is significant to note that DNA in this size range bound to glass only in the presence of high concentration chaotropic salt and remained bound after chaotropic salt removal with washing using 50% aqueous ethanol.

As a general matter, nucleic acid purifications typically rely on ethanol to cause nucleic acids to bind to solid supports for purification purposes. While ethanol has served this purpose well, there is a need in the art for other solvents, which may have complementary or advantageous properties as compared to ethanol. A method that identifies other organic solvents that could be used in nucleic acid purification would allow the user to have other options depending on the need when selecting a protocol for nucleic acid purification.

### SUMMARY OF THE INVENTION

The present invention addresses needs in the art by providing methods, compositions, and kits for identifying organic solvents that can be used in nucleic acid purification. The invention is based, at least in part, on the discovery that certain physical and/or chemical characteristics possessed by ethanol can be applied as a screen to identify other organic solvents that aid in nucleic acid purification. The organic solvents discovered by this method allow nucleic acid purification without organic solvent extractions and ethanol precipitations, and allow separation of single-stranded nucleic acids from double-stranded nucleic acids.

In a first aspect, the invention provides a method of identifying organic solvents that are useful in purifying nucleic acids from samples, such as genomic DNA, total RNA, and mRNA from lysed cells. In general, the method comprises selecting an organic solvent that is miscible or soluble in water and has a dielectric constant of less than 80, and testing the solvent in a nucleic acid purification protocol to determine if it can cause separation of one or more nucleic acids from other substances. In embodiments, the organic solvent is used in conjunction with one or more salts. In preferred embodiments, the organic solvent is useful in causing nucleic acids to bind to a solid support, such as one comprising glass. The method can also comprise not testing organic solvents having a dielectric constant of 80 or higher.

While not being so limited, typically the solvent can be classified as any substance found in Tables 1, 2, and 3, such as an alkane, alcohol, sulfide, organic acid, phosphate, anhydride, ketone, nitrile, cyclic or acyclic ether, sulfoxide, thiophene (e.g., thiophene 1,1-dioxide), amine, ester, amide (aliphatic, cyclic, or heterocyclic), or heterocyclic compounds containing one or more of the same or different heteroatoms (such as morpholine). The solvent can be miscible in water (capable of mixing in any ratio without phase separation), very soluble in water (capable of mixing in a limited ratio without phase separation), or soluble in water (capable of mixing in a limited ratio but with phase separation). For example, the solvent can be one that demonstrates solubility in water of 30% to 80%. Preferably, the solvent in miscible in water. In certain situations, solvents that are immiscible or insoluble in water are excluded by the method. In some situations, one or more alcohols, such as ethanol, propanol, and isobutanol, are excluded.

The general method outlined above can include one or more optional steps. For example, the method can include one or more steps based on whether the solvent is miscible in water, very soluble in water, or soluble in water. In some cases, miscible solvents having dielectric constants of less than 80 can be tested directly for their suitability in nucleic acid purification. In cases where the organic solvent that is selected is miscible in water, direct testing of the organic solvent is performed. In cases where the selected organic solvent is very soluble or soluble in water, the method further comprises determining the solubility: if the organic solvent is miscible in water up to 80% (vol/vol), such as from 30%-80%, then the testing step is performed; if the solvent is less than 30% soluble, the solvent is rejected and not tested.

Identification of the characteristics comprising the method can occur in any order. Therefore, for example, the method can include first determining the dielectric constant of the solvent and then determining the miscibility of the solvent, or vice versa. Any order can be used to determine if an organic solvent exhibits the characteristics useful for a solvent according to the present invention.

Certain chemical modifications can change the miscibility of solvents to improve their usefulness within the context of this invention. For example, a covalent modification to a solvent can convert it from an insoluble solvent to a soluble solvent, a soluble solvent to a very soluble or miscible solvent, and a very soluble solvent to a miscible solvent. If a chemical modification converts a solvent that does not have a desirable characteristics to one that has such a characteristic, and is useful in nucleic acid purification, then the method of the invention can include modifying the solvent prior to identifying it.

The general method of identifying organic solvents according to the present invention includes the optional step of testing solvents for their suitability in nucleic acid purification. That is, the testing step can be any step or series of steps that are suitable for isolation or purification of DNA or RNA from a sample in which it is present. A simple example of such a testing protocol comprises: adding the solvent to an aqueous sample containing the nucleic acid; mixing the solvent with the water; allowing adequate time for the nucleic acid to precipitate from the mixture; and optionally separating the nucleic acid from the mixture (e.g., by centrifugation). Another, somewhat more complex example of a testing protocol comprises differentially isolating DNA and RNA from a sample as follows: separating cultured cells from culture media or separating white blood cells (WBC) from red blood cells and plasma proteins by retaining them on a filter (e.g., a glass fiber filter); washing the filter with phosphate buffered saline (PBS) to remove contaminating proteins and nucleic acids from plasma and/or lysed cells; lysing the cultured cells or WBC retained on the filter with a lysis solution comprising a chaotropic salt and detergent by passing the lysis solution over the filter and cells, resulting in lysis of the cells and the retention of genomic DNA on the filter; mixing the flow through fraction (containing RNA) from the first filter with an organic solvent (and optionally increasing the concentration of chaotropic salt); exposing the mixture to a second filter (e.g., a glass fiber filter comprising one or more filter units) under conditions that allow the RNA in the mixture to bind to the second filter; removing the chaotropic salt by washing the second filter with an aqueous composition, such as one comprising the organic solvent; and eluting RNA from the second filter using low ionic strength buffer or water. This protocol is disclosed in detail in U.S. patent application Ser. Nos. 11/688,652 and 11/688,662, which are hereby incorporated herein in their entireties by reference.

The method of the invention has been used successfully to identify organic solvents that are useful in purifying DNA, RNA, or both from samples, including complex samples comprising various other biological molecules. The organic solvents that are identified by the method can provide purified nucleic acid (e.g., total RNA from mammalian cells) that is of similar or identical yield and similar or identical quality as that purified using similar procedures, but using ethanol as the organic solvent. The method of the present invention thus provides a way of identifying organic solvents that can be used as alternatives to ethanol in nucleic acid purification schemes.

As used herein, organic solvents and water are referred to as “solvent” and “solute”, respectively. While in mixtures of liquids the substance in highest concentration is conventionally referred to as the solvent, herein the organic solvent or organic phase is referred to as the solvent, regardless of its relative concentration in the mixture. Solvents can be characterized by their tendency to form a uniform blend with water called water miscibility. Stated another way, water miscibility is the extent to which a solvent is capable of mixing in any ratio with water without separation into two phases. In terms of the present invention, “miscibility in water” means the solvent is capable of mixing with water in any ratio without phase separation. “Very soluble in water” means that the solvent is capable of mixing in a limited ratio without phase separation and “soluble in water” means that the solvent is capable of mixing in a limited ratio but with phase separation. The degree of miscibility or solubility is employed in the method to identify solvents that are good candidates for use in nucleic acid purification. Non-limiting examples of common solvents that can be considered as soluble, very soluble, and miscible according to their miscibility in water are shown in Table 1, Table 2, and Table 3, respectively.

TABLE 1
Soluble Organic Solvents

| | Dielectric | |
|---|---|---|
| Solvent | Constant (DC) | Class |
| acetal | 3.8 | di-ether |
| aniline | 7.06 | benzenamine |
| benzyl alcohol | 11.92 | OH |
| 1-bromonaphthalene | 4.77 | 2-aro. |
| butanal | 13.45 | aldehyde |
| butane | 1.77 | alkane |
| 1-butanol | 17.84 | OH |
| cis-2-butene-1,4-diol | NV | OH |
| sec-butylamine | NV | amine |
| carbon disulfide | 2.63 | sulfide |
| chloromethane | 10 | alkane |
| o-cresol | 6.76 | Aro(6) OH |
| crotonaldehyde (trans) | NV | aldehyde |
| cyclohexanol | 16.4 | OH |
| cyclohexanone | 16.1 | ketone |
| cyclohexylamine | 4.55 | amine |
| dibutylamine | 2.77 | amine |
| dichlorodifluoromethane | 3.5 | alkane |
| diethylene glycol | 31.82 | OH ether |
| 2,3-dimethyl-2-butanol | NV | OH |
| dimethylether | 6.18 | ether |
| dipropylamine | 3.07 | amine |
| ethyl acetate | 6.08 | ester |
| ethylene glycol dimethylether | 7.3 | ether |
| ethyl formate | 8.57 | ester |
| furfural | 42.1 | ether-ald. |
| hexylene glycol | 23.4 | OH |
| isobutanal | NV | aldehyde |
| isopropyl acetate | NV | ester |
| mesityl oxide | 15.6 | ketone |
| methacrylic acid | NV | acid |
| 3-methyl butanoic acid | NV | acid |
| 2-methyl-2-butanol | 5.78 | OH |
| 2-methyl-tetrahydrofuran | 6.97 | ether |
| nitromethane | 37.27 | alkane |
| 1,5-pentanediol | 26.2 | OH |
| pentanoic acid | 2.66 | acid |
| phenol | 12.4 | benz.-OH |
| phenyl ethylamine | NV | amine |
| propanal | 18.5 | aldehyde |
| propane | 1.67 | alkane |
| propargyl alcohol | 20.8 | OH |
| propylamine | 5.08 | amine |
| tributylphosphate | 8.34 | PO4 |
| triethylamine | 2.42 | amine |
| triethyl phosphate | 13.2 | PO4 |
| trifluoroacetic acid | 8.42 | acid |
| 2,4,6-trimethylpyridine | 7.81 | N-het.(6) |
| 2,5-xylenol | 5.36 | OH |
| 2,6-xylenol | 4.9 | OH |
| 3,5-xylenol | 9.06 | OH |

NV = no value available
Aro = aromatic
(5) = 5-membered ring
(6) = 6-membered ring

TABLE 2
Very Soluble Organic Solvents

| | Dielectric | |
|---|---|---|
| | Constant | |
| Solvent | (DC) | Class |
| acetamide | 67.6 | amide |
| acetic anhydride | 22.45 | anhydride |
| acetylacetone | 26.52 | ketone |
| acrolein | NV | aldehyde |
| 2-butanol | 17.26 | OH |
| 2-butene-1,4,diol (trans) | NV | OH |
| caprolactam (epsilon) | NV | lactam |
| chlorodifluoromethane | 6.11 | alkane |
| crotonyl alcohol (cis & trans) | NV | OH |
| trans-crotonoic acid | NV | acid |
| diethanolamine | 25.75 | amine |
| diethylamine | 3.68 | amine |
| diethylene glycol diethyl ether | 5.7 | ether |
| diethylene glycol monoethyl | NV | ether-ester |
| ether acetate | | |
| diethylketone | 17 | ketone |
| dimethylamine | 5.26 | amine |
| ethylacetoacetate | 14 | ester |
| ethylenediamine | 13.82 | amine |
| ethylene glycol diacetate | NV | ester |
| ethylene glycol dibutylether | NV | ether |
| ethylene glycol ethylether | 7.57 | ether-ester |
| acetate | | |
| ethylene glycol | 8.25 | ether-ester |
| monomethylether acetate | | |
| ethylene glycol monoethylether | 13.38 | ether-OH |
| ethyl lactate | 15.4 | ester-OH |
| 1,2,6-hexanetriol | 31.5 | OH |
| 2,4-lutidine | 9.6 | N-het. Aro. |
| methylacetate | 7.07 | ester |
| methylacetoacetate | NV | ester |
| methylamine | 16.7 | amine |
| methylethylketone | 18.56 | ketone |
| methyl formate | 9.2 | ester |
| methyl pentyl ketone | 11.95 | ketone |
| 2-methyl propanoic acid | 2.58 | acid |
| N-methyl-2-pyrrolidone | 32.2 | N-ketone |
| 2-pentanol | 13.71 | OH |
| 2-picoline | 10.18 | N-het.(6) |
| propanenitrile | 29.7 | nitrile |
| propylene carbonate | 66.14 | keto-ether |
| 2-pyrrolidone | NV | N—OH(5) |
| succinonitrile | 62.6 | nitrile |
| trichloroacetic acid | 4.6 | acid |
| tetraethyleneglycol | 20.44 | OH |
| trimethylamine | 2.44 | amine |
| trimethylphosphate | 20.6 | PO4 |

NV = no value available
Aro = aromatic
(5) = 5-membered ring
(6) = 6-membered ring

TABLE 3
Miscible Organic Solvents

| Solvent | Dielectric Constant (DC) | Class |
|---|---|---|
| acetaldehyde | 21 | aldehyde |
| acetic acid | 6.2 | acid |
| acetone | 21.01 | ketone |
| acetonitrile | 36.64 | nitrile |
| acrylic acid | NV | acid |
| allyl alcohol | 19.7 | OH |
| allylamine | NV | amine |
| 2-amino-isobutanol | | OH |
| 1,3-butanediol | 28.8 | OH |
| 1,4-butanediol | 31.9 | OH |
| 2,3-butanediol | NV | OH |
| butanoic acid | 2.98 | acid |
| butylamine | 4.71 | amine |
| t-butylamine | NV | amine |
| diacetone alcohol | 18.2 | OH |
| 1,3-dioxolane | NV | ether |
| 1,4-dioxane | 2.22 | ether |
| dimethylformamide | 38.25 | amide |
| diethyleneglycol dimethylether | NV | ether |
| diethyleneglycol | NV | ether-OH |
| monoethylether | | |
| diethyleneglycol | NV | ether-OH |
| monomethylether | | |
| diethylenetriamine | 12.62 | amine |
| N,N-dimethylacetamide | 38.85 | amide |
| dimethylsulfoxide | 47.24 | sulfoxide |
| ethanol | 25.3 | OH |
| ethanolamine | 31.94 | amine-OH |
| ethylamine | 8.7 | amine |
| ethylene chlorohydrin | 25.8 | OH |
| ethyleneglycol | 41.4 | OH |
| ethyleneglycol monobutyl ether | 9.3 | ether-OH |
| ethyleneglycol monomethyl | 17.2 | ether-OH |
| ether | | |
| ethyleneimine | 18.3 | imine |
| formic acid | 51.1 | acid |
| furfuryl alcohol | 16.85 | OH |
| glycerol | 46.53 | OH |
| hydracrylonitrile | NV | nitrile-OH |
| isobutylamine | 4.43 | amine |
| isopropylamine | 5.63 | amine |
| 2,6-lutidine | 7.33 | N-het. Aro. |
| methanol | 33 | OH |
| 2-methyl-2-propanol | 12.47 | OH |
| morpholine | 7.42 | O—N het. |
| pentylamine | 4.27 | amine |
| 3-picoline | 11.1 | N-het. Aro. |
| 4-picoline | 12.2 | N-het. Aro. |
| piperidine | 4.33 | N-het.(6) |
| 1,2-propanediol | 27.5 | OH |
| 1,3-propanediol | 35.1 | OH |
| propanoic acid | 3.44 | acid |
| 1-propanol | 20.8 | OH |
| 2-propanol | 20.18 | OH |
| pyridine | 13.26 | N-het. Aro. |
| pyrrolidine | 8.3 | N-het(5) |
| sulfolane | 43.26 | S-Diox. |
| tetraglyme | NV | ether |
| tetrahydrofuran | 7.52 | ether |
| 2,2′-thiodiethanol | 28.61 | S ether-OH |
| triethanolamine | 29.36 | amine-OH |
| triethyleneglycol | 23.69 | ether-OH |

NV = no value available
Aro = aromatic
(5) = 5-membered ring
(6) = 6-membered ring

Certain organic solvents from among those listed in Tables 1-3 were tested using the method of identification according to the present invention. Some of those tests are reported in the Examples, below. Included among the many solvents that are suitable as replacements for ethanol in purification schemes, without compromising RNA yield and purity, are: acetone, acetonitrile, 1,4-dioxolane, tetra(ethylene glycol)dimethyl ether, 1,3-dioxolane, diethyleneglycol dimethylether, dimethylsulfoxide (DMSO), sulfolane, tetraglyme, tetrahydrofuran, N-methyl-2-pyrrolidone, and benzyl alcohol. Testing showed that formamide (DC 111) and the organic acid trichloroacetic acid (TCA; DC=4.6) did not yield nucleic acid (RNA) under standard conditions.

The dielectric constants (DC) of the solvents, if known, are also shown in Tables 1, 2, and 3. Dielectric constant is the relative measure of the polarity of a solvent. A high DC correlates to a high polarity, while a low DC correlates to low polarity. DC values presented in the Tables were obtained from the Handbook of Organic Solvents (CRC Press LLC, David R. Lide, ed., Boca Raton, Fla., 1995). It is interesting to note that despite a wide range of DC values in each water solubility category, the average DC for “miscible” (DC of about 23; Table 3) and “very soluble” solvents (DC of about 28; Table 2) are about twice as high as the average DC for “soluble” solvents (DC of 12; Table 1).

While not limited to only those organic solvents tested, the solvents that were found to be the best at nucleic acid purification were primarily found in the miscible group (Table 3). These exemplary solvents are shown in Table 4 along with their dielectric constants and the chemical class of each. Table 4 also includes the DC values for ethanol and water. Interestingly, the majority of the solvents included in this table have relatively high DC values. Also of note, the method of the present invention has identified a variety of solvents with functional groups different from the hydroxyl group of ethanol. These different functional groups include alcohol, nitrile, ketone, acyclic and cyclic ether, sulfoxide, and thiophene 1,1-dioxide.

TABLE 4
Exemplary Solvents For Nucleic Acid Purification

| Solvent | Dielectric Constant | Class |
|---|---|---|
| acetonitrile | 36.64 | nitrile |
| acetone | 21.01 | ketone |
| tetrahydrofuran | 7.52 | cyclic ether |
| sulfolane | 43.26 | thiophene 1,1- |
| | | dioxide |
| 1,3-dioxolane | NV | cyclic ether |
| tetraglyme | NV | acyclic ether |
| dimethyl sulfoxide | 47.2 | sulfoxide |
| ethanol | 25.3 | alcohol |
| water | 80.0 | hydride |

NV = no value available

Although any nucleic acid purification testing scheme may be used in accordance with the present invention, for identification of the organic solvents listed in the tables above, the solvents were tested in either the Stratagene Absolutely RNA® Miniprep kit or in a nucleic acid purification (NAP) protocol to determine their effectiveness. Either cultured Jurkat cells or blood was used as samples comprising the nucleic acid of interest. In general, the NAP protocol for purifying both genomic DNA and total-RNA (RNA) from mammalian cells took advantage of chaotropic salts, glass fiber filter (GF), and organic solvents. Unique features of this protocol are: (1) cultured cells are separated from culture media or white blood cells (WBC) are separated from red blood cells (RBC) and serum proteins on glass fiber filters; (2) cultured cells or WBC are lysed with high concentration chaotropic salt plus detergent and genomic DNA is quantitatively retained on GF, even after water removal of chaotropic salt and detergent in the absence of organic solvent; (3) cultured cells or WBC genomic DNA are recovered by low ionic strength buffer or water “back-flow” through the GF; (4) RNA flowing through (FT) the first GF containing high concentration chaotropic salt binds to a second GF by mixing the FT with a wide variety of organic solvents; (5) RNA recovery from GF is accomplished by removal of the chaotropic salt with aqueous organic solvent followed by elution in high yield and purity with low ionic strength buffer or water.

The purification protocol, using whole blood as a representative sample, involves sample passage through two GF resulting in blood cell collection. RBC are lysed on the GF and RBC contents plus plasma proteins are removed by washing the GF with an isotonic solution such as phosphate buffered saline (PBS). PBS maintains WBC integrity while these cells are trapped on/in the GF. WBC lysis solution, containing detergent and high concentration chaotropic salt, is passed through the GF resulting in WBC lysis. Virtually all genomic DNA is trapped on/in the GF while RNA in WBC lysis solution flows through the GF into a collection chamber (FT). An equal volume of 70 to 100% of the identified organic solvent to be tested is added to the FT and the resulting mixture passed through a second set of GF to which RNA binds. Genomic DNA and RNA are then recovered from GF as described above in steps (3) and (5), respectively.

In another aspect, the invention provides compositions. In general, the compositions comprise one or more organic solvents that can be identified by the method described above, and at least one other substance. Typically, the compositions are useful in purification of nucleic acids from samples. Accordingly, the compositions typically comprise one or more of the following substances: water; nucleic acids (DNA, RNA, or mixtures of both); proteins, polypeptides, peptides; polysaccharides; lipids; salts; minerals; other organic solvents; buffers; and nucleic acid binding agents (e.g., solid supports, such as those comprising glass, metals, or nylon or other man-made substances). Typically, the composition comprises one or more substances found in cells, cell lysates, or nucleic acid purification or analysis procedures.

In exemplary embodiments, the composition comprises an organic solvent identifiable by the present method, and one or more salts. For example, for RNA purification the composition may comprise an organic solvent at a concentration of 10%-80% and a chaotropic salt at a concentration of 1-8 Molar. The composition may, in embodiments, comprise an organic solvent, one or more salts and nucleic acid. Thus, the composition may comprise an organic solvent, one or more salts and DNA, RNA, or a mixture of DNA and RNA. Often, the composition will be created as part of a nucleic acid purification scheme, and will comprise nucleic acid (preferably RNA), a chaotropic salt, and the organic solvent. In some embodiments, the composition comprises a solid support, such as a glass fiber filter, which is either unbound or bound by a nucleic acid (e.g., RNA). In some embodiments, the composition does not comprise an alcohol, such as ethanol, isopropanol, or isobutanol.

In an additional aspect, the invention provides kits comprising one or more containers that independently contain an organic solvent that can be identified according the method of the present invention, and one or more substances that are useful in purification of nucleic acids. For example, the kit may comprise an organic solvent and one or more glass fiber filters. Likewise, the kit may comprise an organic solvent and a chaotropic salt. Other non-limiting exemplary components of the kit include: a mineral support of any composition, one or more cell lysis solutions, wash solutions, elution solutions, or two or more of these in combination. The kits can be used, for example, to isolate biological molecules, such as nucleic acids. In general, the kits comprise some or all of the materials, reagents, supplies, etc. needed for isolating nucleic acids from samples. Thus, in various embodiments, the kit may comprise organic solvents, one or more buffers such as cell lysis buffers, DNase, DNase reconstitution buffer, DNase digestion buffer, RNase, RNase reconstitution buffer, RNase digestion buffer, high salt wash buffer, low salt wash buffer, and/or elution buffer. The kit may likewise comprise columns, such as prefiltration columns to filter the sample, columns to adsorb nucleic acid molecules, and/or columns to purify proteins.

### BRIEF DESCRIPTION OF THE DRAWINGS

The accompanying drawings, which constitute a part of this specification, illustrate several embodiments of the invention and, together with the written description, serve to explain various principles of the invention. It is to be understood that the drawings are not to be construed as a limitation on the scope or content of the invention.

FIG. 1 depicts the quality of RNA isolated from Jurkat cells using ethanol as a solvent, as seen by data from an Agilent Bioanalyzer.

FIG. 2 depicts the quality of RNA isolated from white blood cells using ethanol as a solvent, as seen by data from an Agilent Bioanalyzer.

FIG. 3 depicts the quality of RNA isolated from Jurkat cells using acetone as a solvent as seen by data from an Agilent Bioanalyzer.

FIG. 4 depicts the quality of RNA isolated from Jurkat cells comparing ethanol and acetone as solvents as seen by data from QRT-PCR using a Stratagene Mx 3000P Real-Time PCR instrument.

FIG. 5 depicts the quality of RNA isolated from Jurkat cells using acetonitrile as a solvent as seen by data from an Agilent Bioanalyzer.

FIGS. 6A, 6B, and 6C depict the quality of RNA isolated from white blood cells comparing acetonitrile and ethanol as solvents as seen by data from QRT-PCR using a Stratagene Mx 3000P Real-Time PCR instrument.

FIG. 7 depicts the quality of RNA isolated from Jurkat cells using tetraglyme as a solvent as seen by data from an Agilent Bioanalyzer.

FIGS. 8A and 8B depict the quality of RNA isolated from Jurkat cells comparing ethanol and tetrahydrofuran as solvents, respectively, as seen by data from an Agilent Bioanalyzer.

FIG. 9 depicts the quality of RNA isolated from Jurkat cells comparing ethanol and tetrahydrofuran as solvents as seen by data from QRT-PCR using a Stratagene Mx 3000P Real-Time PCR instrument.

FIGS. 10A and 10B depict the quality of RNA isolated from white blood cells comparing ethanol and tetrahydrofuran as solvents, respectively, as seen by data from an Agilent Bioanalyzer.

FIGS. 11A, 11B, and 11C depict the quality of RNA isolated from white blood cells comparing ethanol and tetrahydrofuran as solvents, as seen by data from QRT-PCR using a Stratagene Mx 3000P Real-Time PCR instrument.

FIGS. 12A, 12B, 12C, and 12D depict the quality of RNA isolated from Jurkat cells comparing ethanol, 45% sulfolane, 40% sulfolane and 35% sulfolane, respectively, as solvents, as seen from data from an Agilent Bioanalyzer.

FIG. 13 depicts the quality of RNA isolated from Jurkat cells using 1,3-dioxolane as a solvent, as seen from data from an Agilent Bioanalyzer.

FIG. 14 depicts the quality of RNA isolated from Jurkat cells comparing ethanol, dimethylsulfoxide (DMSO), and formamide as solvents, as measured by Nanodrop UV spectrophotometry.

### EXAMPLES

The invention will be further explained by the following Examples, which are intended to be purely exemplary of the invention, and should not be considered as limiting the invention in any way.

### Example 1

### Ethanol as an Organic Solvent in Nucleic Acid Purification

RNA was isolated from a Jurkat cell line using the following protocol. Cultured cells (2×10⁷) were collected in a centrifuge tube and washed with PBS buffer (GIBCO formulation). The cells were resuspended in 10 ml of PBS and passed through two GF/D filters (47 mm diameter each) to capture the cells. The filters were washed with 20 ml of PBS to further reduce contaminants. Nine ml of White Blood Cell (WBC) Lysis Solution (4 M guanidine thiocyanate, 1% Triton X-100, 0.05% sarkosyl, 0.01% Antifoam A, 0.7% beta-mercaptoethanol) was passed through the filters resulting in the release of nucleic acids from the cells and the lysate was collected comprising mostly RNA. The genomic DNA was retained on the GF/D filters and could be physically and/or chemically retrieved later. Four ml of water was passed through the GF/D filters to release additional RNA and this fraction was added to the WBC lysate. Ethanol was adjusted to a final concentration of 35%. The resulting mixture was passed over five GF/F filters (9.5 mm diameter each). The GF/F filters were washed three times with 2.5 ml of Low Salt Wash Solution (2 mM Tris (pH 6-6.5), 20 mM NaCl, 80% ethanol) for a total of 7.5 ml. The filters were purged of excess liquid between each addition of Low Salt Wash Solution and after the final addition, the filters were air dried. The RNA was eluted from the GF/F filters with 100 ul (microliters) of RNase-free water. The eluted RNA was checked for yield by measuring absorbance on a spectrophotometer at A₂₆₀ and purity was checked using the A₂₆₀/A₂₈₀ ratio.

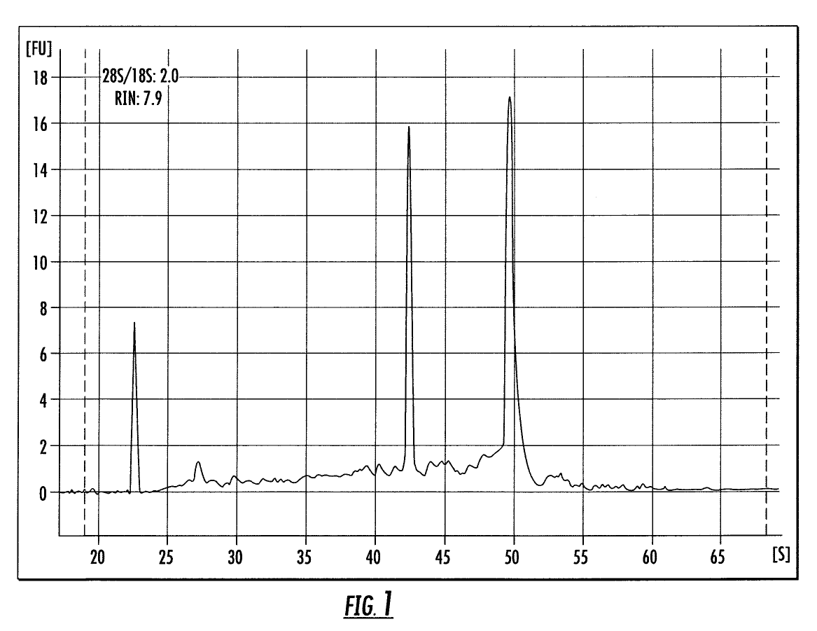

Results from this experiment can be seen in Table 5 and FIG. 1. Thirty-five percent (35%) ethanol in the binding buffer allowed purification of RNA as shown by the amount of RNA recovered from the filters. Agilent Bioanalyzer traces demonstrated that 35% ethanol in the binding buffer resulted in good quality RNA as seen by a 28S/18S ratio of 2.0 and a RIN of 7.9 (depicted graphically in FIG. 1).

TABLE 5
Results of Purification of RNA Using 35% Ethanol

| Sample | RNA (ng/ul) | A260/280 |
|---|---|---|
| 1 | 65.81 | 2.1 |
| 2 | 67.3 | 2.09 |

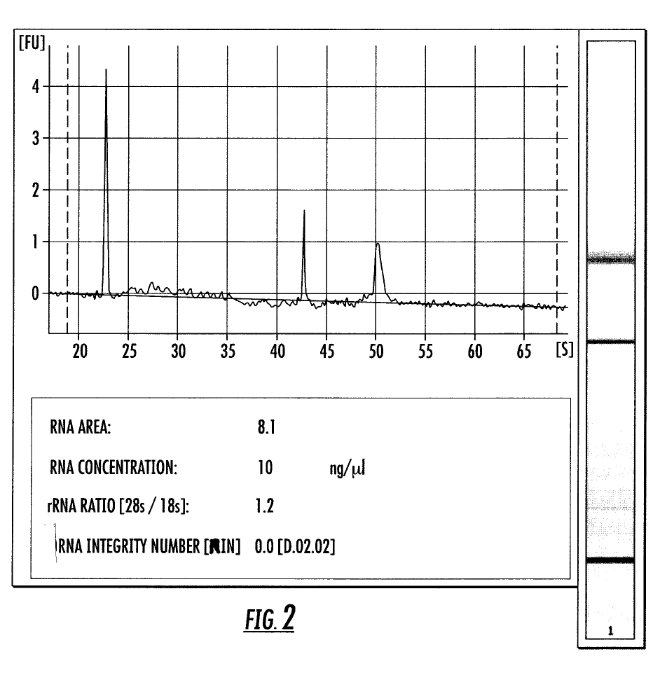

RNA was purified from white blood cells using a modification of the protocol described above for purification of RNA from cultured cells (called Nucleic Acid Purification or NAP protocol). Five milliliters of blood, collected in a vacutainer tube with EDTA anticoagulant, was mixed with 20 ml Red Cell Lysis Solution (0.15 M ammonium chloride, 0.001 M potassium bicarbonate, 0.0001 M EDTA, pH 7.2-7.4) and incubated at room temperature for 5 minutes. White blood cells were collected by centrifugation and processed starting at the PBS buffer step as described above. Analysis of the RNA by UV spectrophotometry showed good RNA yield and purity (Table 6). Agilent Bioanalyzer traces of the purified RNA also showed good quality of the RNA isolated using 35% ethanol as seen by a 28S/18S ratio of 1.2 and a RIN of 8.9 (depicted in FIG. 2).

TABLE 6
Purification of RNA Using Ethanol

| Sample | RNA (ng/ul) | A260/280 |
|---|---|---|
| 1 | 12.26 | 2.04 |
| 2 | 9.87 | 2.24 |

RNA from white blood cells can also be isolated using the Stratagene Absolutely RNA® kit, which employs spin cups comprising a silica-based fiber matrix (called spin-cup protocol). White blood cells are collected from 5 ml of blood as described above. After transfer of the cells to a microcentrifuge tube, the cells are collected in a loose pellet by spinning at a low speed for 5 min. The supernatant is discarded. White Blood Cell Lysis Solution (600 ul) is added and the sample is homogenized by vortexing or repeated pipetting. The homogenate (700 ul) is transferred to a Prefilter Spin Cup and spun in a microcentrifuge at maximum speed for 5 min. The filtrate is retained and ethanol is added to a final concentration of 35%. The mixture is vortexed for 5 sec and transferred to an RNA Binding Spin Cup. The tube is spun in a microcentrifuge at maximum speed for 30-60 sec. The filtrate is discarded and the spin cup is washed with 600 ul of Low-Salt Wash Buffer (2 mM Tris, pH 6-6.5, 20 mM NaCl, 80% ethanol). DNase in a digestion buffer (10 mM Tris, pH 7.5, 50% glycerol) is added to the spin cup and incubated at 37° C. for 15 min. The spin cup is washed with 600 ul of High-Salt Wash Buffer (2 M guanidine thiocyanate, 50 mM Tris, pH 6.4, 40% ethanol) followed by two washes (600 ul and 300 ul) with the Low-Salt Wash Buffer. The spin cup is spun once more to dry the fiber matrix. Elution buffer (100 ul; 0.01 M Tris pH 7.5, 0.0001 M EDTA) is added to the spin cup to elute the RNA from the fiber matrix. As described previously, analysis of the RNA can be performed by UV spectrophotometry to show RNA yield and purity, and Agilent Bioanalyzer traces and Quantitative Real Time PCR (QRT-PCR) of the purified RNA can be used to show the quality of nucleic acid.

### Example 2

### Acetone as an Organic Solvent in Nucleic Acid Purification

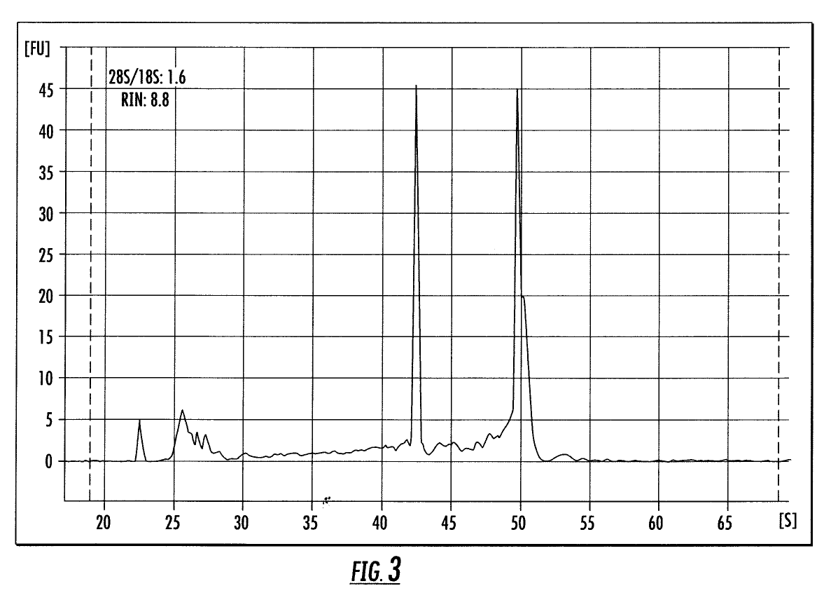

RNA was isolated from a Jurkat cell line using the same protocol as described in Example 1 with the exception that acetone was used as a solvent in the binding buffer instead of ethanol. Acetone was added to the binding buffer at a final concentration of 33%, 50%, and 66% to determine the effect of different concentrations of acetone on RNA yield and purity. An ethanol control using a final concentration of 50% ethanol was also performed. As shown in Table 7, 33% and 50% acetone resulted in good yields of RNA, but 66% acetone lead to low yields of RNA recovery. Agilent Bioanalyzer traces of the 33% acetone sample showed good quality of RNA (depicted in FIG. 3) compared to ethanol.

TABLE 7
Comparison of RNA Purity Using Ethanol and Acetone

| Acetone (%) | Ethanol (%) | RNA (ng/ul) | A260/280 |
|---|---|---|---|
| — | 50 | 54 | 2.03 |
| 33 | — | 70 | 2.06 |
| 50 | — | 60 | 2.04 |
| 66 | — | 6 | 1.32 |

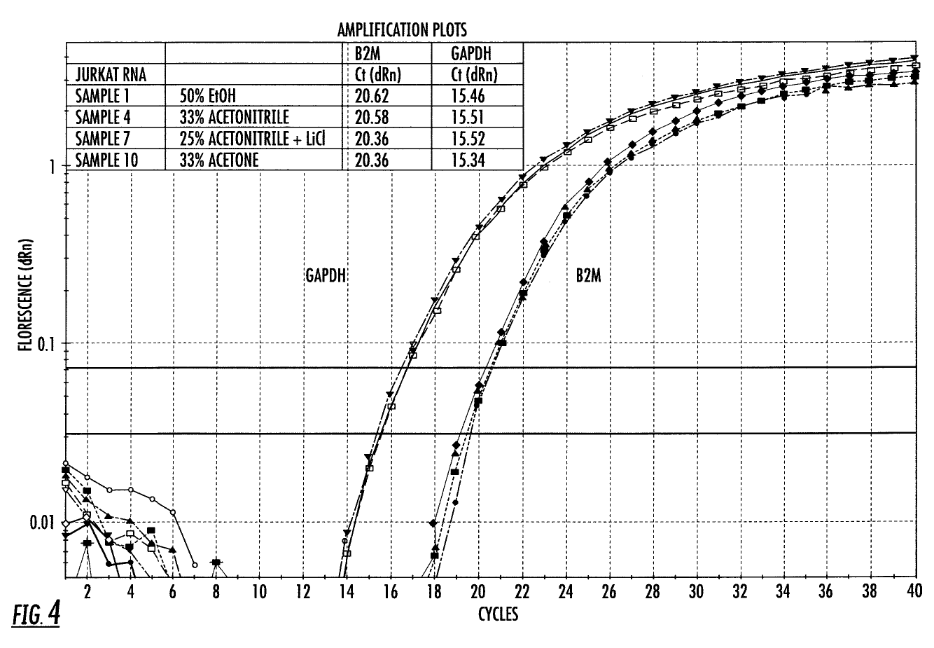

In some examples, RNA quality was also analyzed by reverse transcription and amplification of beta-2-microglobulin (B2M), glyceraldehyde-3-phosphate dehydrogenase (GAPDH), beta-globin, and/or alpha-1 antitrypsin (alpha-1AT) mRNA using Quantitative Real Time PCR (QRT-PCR). In general, QRT-PCR reactions were performed using 10 ng of each RNA (25 ul reaction volume), Brilliant QRT-PCR Master Mix,1-step (Stratagene) and TaqMan primers and probe (B2M, GAPDH and alpha-1AT, Assay on Demand, ABI) and beta-globin TaqMan primers and probe set (beta-globin sense primer 5′-TGCACGTGGATCCTGAGAACT-3′ (SEQ ID NO:1), beta-globin anti-sense primer 5′-AATTCTTTGCCAAAGTGATGGG-3′ (SEQ ID NO:2), 5′-FAM/CAGCACGTTGCCCAGGAGCCTG/3BHQ_{—}1/-3′ (SEQ ID NO:3) on the Mx3000P Real-Time PCR System (Stratagene) using the following cycling parameters: 50°/30 min, then 95°/10 min followed by 40 cycles of 95°/15 sec; 60°/1 min. In this example, FIG. 4 shows plots of QRT-PCR reactions that amplified GAPDH and B2M. The acetone and ethanol samples showed similar Cts for the tested genes and amplification curves that overlapped, showing that the RNA samples had an equal quality.

### Example 3

### Acetonitrile as an Organic Solvent in Nucleic Acid Purification

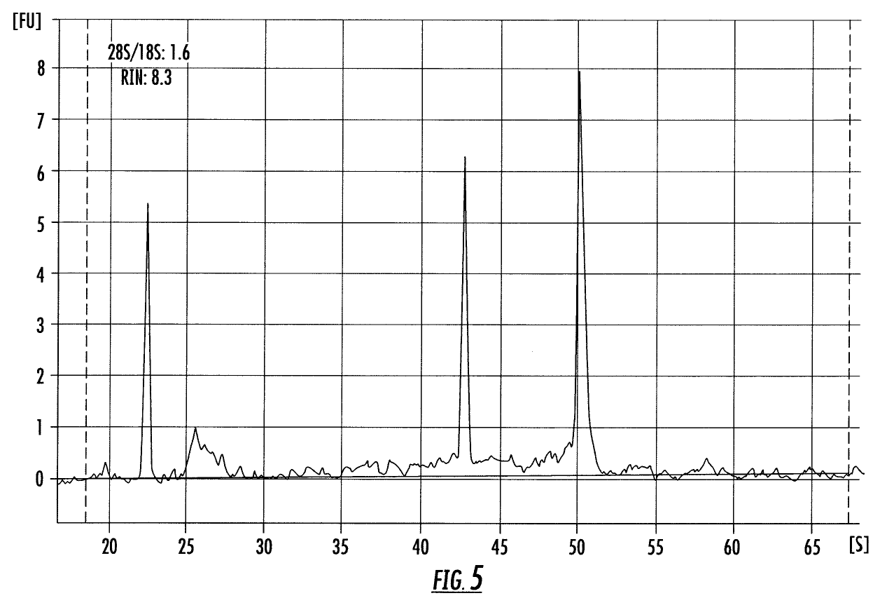

RNA was isolated from a Jurkat cell line using the same protocol as described in Example 1 with the exception that acetonitrile was used as a solvent in the binding buffer instead of ethanol. Acetonitrile was added to the binding buffer at a final concentration from 20% to 66% to determine the effect of different concentrations of acetonitrile on RNA yield and purity. Ethanol was added to the binding buffer at a final concentration of 50% as a control. As shown in Table 8, good yield of RNA was found when using a range of 25% to 40% acetonitrile in the binding buffer. Optimal yield and quality was seen at 25% acetonitrile in the binding buffer. QRT-PCR experiments that amplified GAPDH and B2M showed similar Cts using the acetone, acetonitrile, and ethanol samples and overlapping amplification curves, suggesting that all the RNA samples had an equal quality (depicted in FIG. 4). Agilent Bioanalyzer traces of the 33% acetonitrile sample showed good quality of RNA as seen by a 28S/18S ratio of 1.6 and a RIN ratio of 8.3 (depicted in FIG. 5).

TABLE 8
Comparison of RNA Purity Using Ethanol and Acetonitrile

| Acetonitrile | | RNA | |
|---|---|---|---|
| (%) | Ethanol (%) | (ng/ul) | A260/280 |
| — | 50 | 54 | 2.03 |
| 20 | — | 46 | 2 |
| 25 | — | 80 | 2.28 |
| 33 | — | 64 | 2 |
| 40 | — | 68 | 2.07 |
| 50 | — | 19 | 1.74 |
| 66 | — | 11 | 2.26 |

RNA was also isolated from white blood cells using both the NAP and spin-cup protocols described in Example 1 with the exception that acetonitrile was used as a solvent in the binding buffer instead of ethanol. In the NAP experiments, acetonitrile was added at a final concentration of 9% to 50% to determine the effect of using different concentrations of acetonitrile in the binding buffer on isolation of RNA and in the spin-cup experiments, acetonitrile was added at a final concentration of 16% to 50%. Ethanol was added to the binding buffer at a final concentration of 50% as a control. As can be seen from Table 9, the highest yield of RNA from the NAP procedure was found at a final acetonitrile concentration of 44%. The highest yield of RNA from the spin-cup procedure was found from samples containing acetonitrile in the range of 28% to 37% (Table 10).

TABLE 9
RNA Yield Using Acetonitrile Or Ethanol In NAP Protocol

| Acetonitrile | | RNA | |
|---|---|---|---|
| (%) | Ethanol (%) | (ng/ul) | A260/280 |
| — | 50 | 2.0 | 1.82 |
| 9 | — | 2.9 | 1.15 |
| 16 | — | 4.12 | 1.15 |
| 23 | — | 3.3 | 1.23 |
| 28 | — | 3.56 | 1.57 |
| 33 | — | 4.68 | 1.52 |
| 37 | — | 4.68 | 1.42 |
| 44 | — | 8.07 | 1.6 |
| 50 | — | 3.79 | 2.52 |

TABLE 10
RNA Yield Using Acetonitrile Or Ethanol In Spin Cup Protocol

| Acetonitrile | | RNA | |
|---|---|---|---|
| (%) | Ethanol (%) | (ng/ul) | A260/280 |
| — | 50 | 8.06 | 1.21 |
| 16 | — | 8.60 | 1.84 |
| 23 | — | 10.62 | 1.73 |
| 28 | — | 12.12 | 1.68 |
| 33 | — | 11.73 | 1.8 |
| 37 | — | 12.09 | 1.8 |
| 44 | — | 8.94 | 1.78 |
| 50 | — | 5.66 | 1.67 |

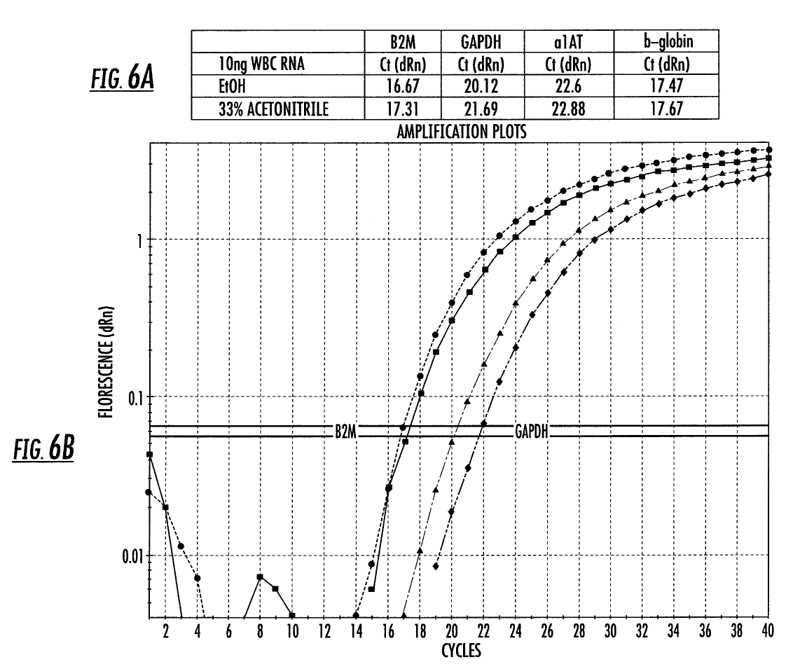

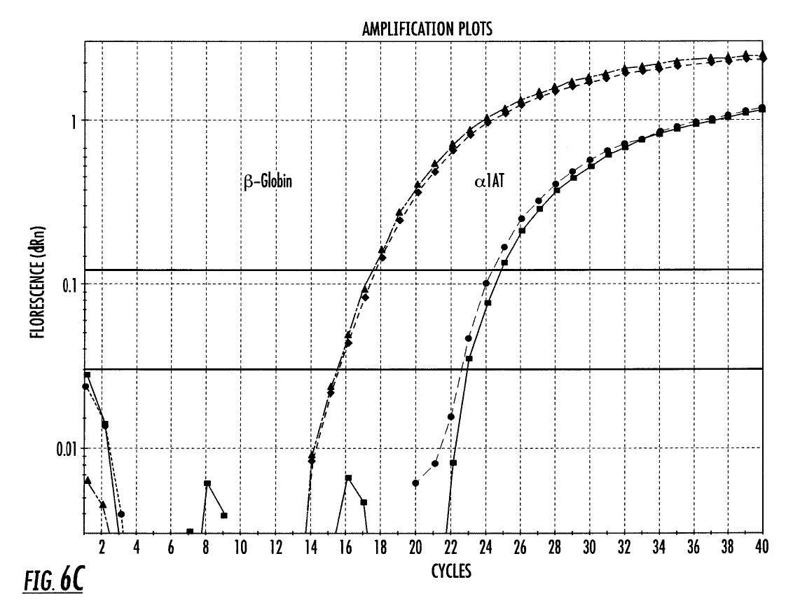

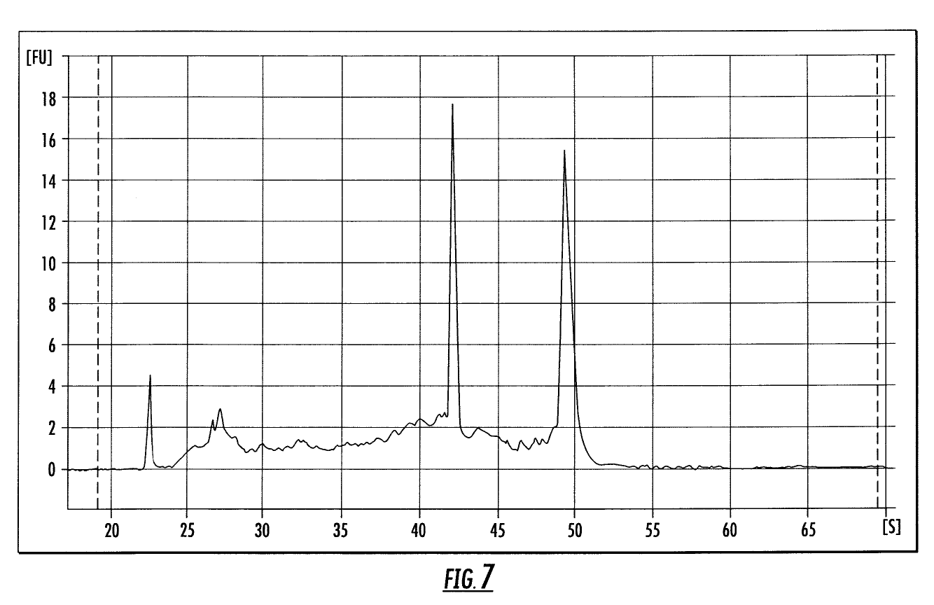

QRT-PCR experiments that amplified GAPDH, B2M, beta-globin, and alpha-1AT showed similar Cts between the acetonitrile and ethanol samples and overlapping amplification curves, suggesting that the RNA samples had an equal quality (see FIG. 6, Panels A-C).

### Example 4

### Tetraglyme as an Organic Solvent in Nucleic Acid Purification

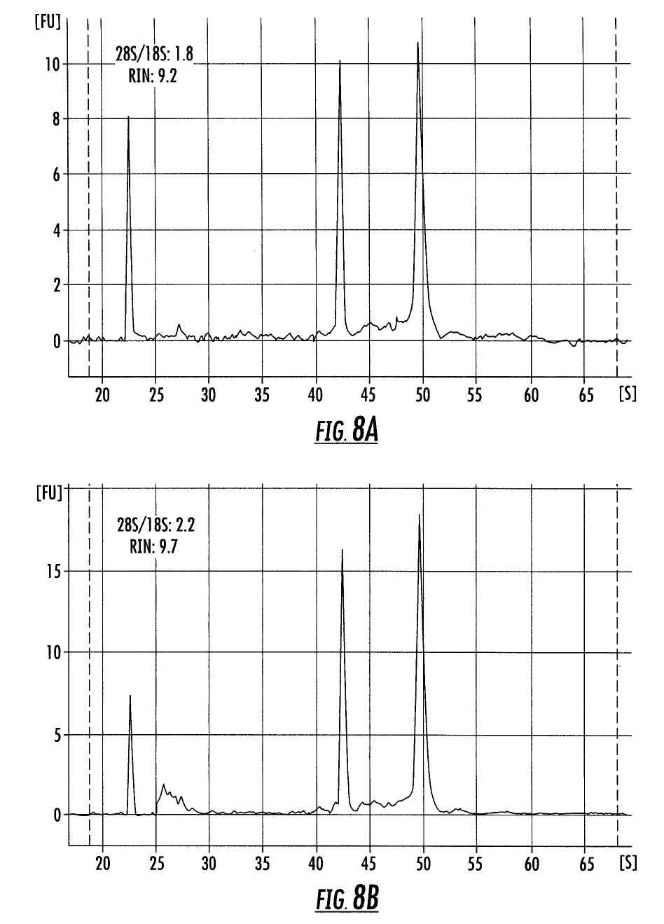

RNA was isolated from a Jurkat cell line using the same protocol as described in Example 1 with the exception that tetraglyme was used as a solvent in the binding buffer instead of ethanol. Tetraglyme was added to the binding buffer at a final concentration ranging from 30% to 45% to determine the effect of different concentrations of tetraglyme on RNA yield and purity. An ethanol sample at a final concentration of 35% was used in the experiment as a control. As shown in Table 11, 30% tetraglyme in the binding buffer resulted in the highest yield of RNA. Agilent Bioanalyzer traces of the 30% tetraglyme sample showed good quality of RNA as seen by a 28S/18S ratio of 1.9 and a RIN ratio of 6.8 (see FIG. 7).

TABLE 11
RNA Yield Using Tetraglyme Or Ethanol In Binding Mixture

| Tetraglyme | | RNA | |
|---|---|---|---|
| (%) | Ethanol (%) | (ng/ul) | A260/280 |
| — | 35 | 75.95 | 2.15 |
| 30 | — | 91.8 | 2.04 |
| 35 | — | 58.69 | 2.02 |
| 40 | — | 65.44 | 2.05 |
| 45 | — | 59.87 | 2.06 |

### Example 5

### Tetrahydrofuran as an Organic Solvent in Nucleic Acid Purification

RNA was isolated from a Jurkat cell line using the same protocol as described in Example 1 with the exception that tetrahydrofuran was used as a solvent in the binding buffer instead of ethanol. Tetrahydrofuran was added to the binding buffer at a final concentration from 25% to 40% to determine the effect of different concentrations of tetrahydrofuran on RNA yield and purity. Ethanol was added to the binding buffer at a final concentration of 35% as a control. As shown in Table 12, the best yield of RNA was found in the 30% to 40% tetrahydrofuran samples.

TABLE 12
RNA Yield Using Tetrahydrofuran Or Ethanol In Binding Mixture

| Tetrahydrofuran | Ethanol | RNA | |
|---|---|---|---|
| (%) | (%) | (ng/ul) | A260/280 |
| — | 35 | 31.8 | 2.05 |
| 25 | — | 33.53 | 2.1 |
| 30 | — | 42.4 | 2.12 |
| 40 | — | 43.4 | 2.13 |

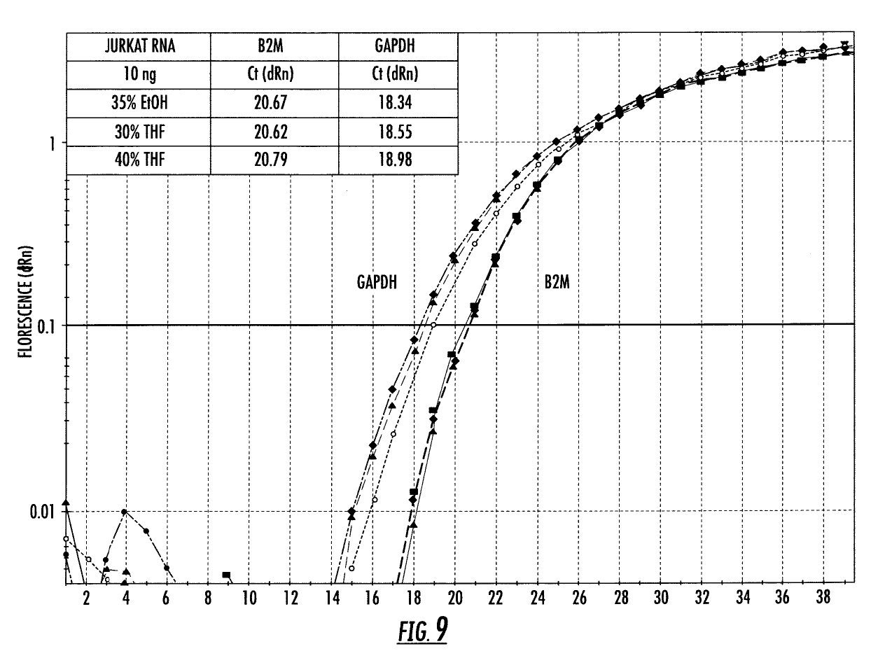

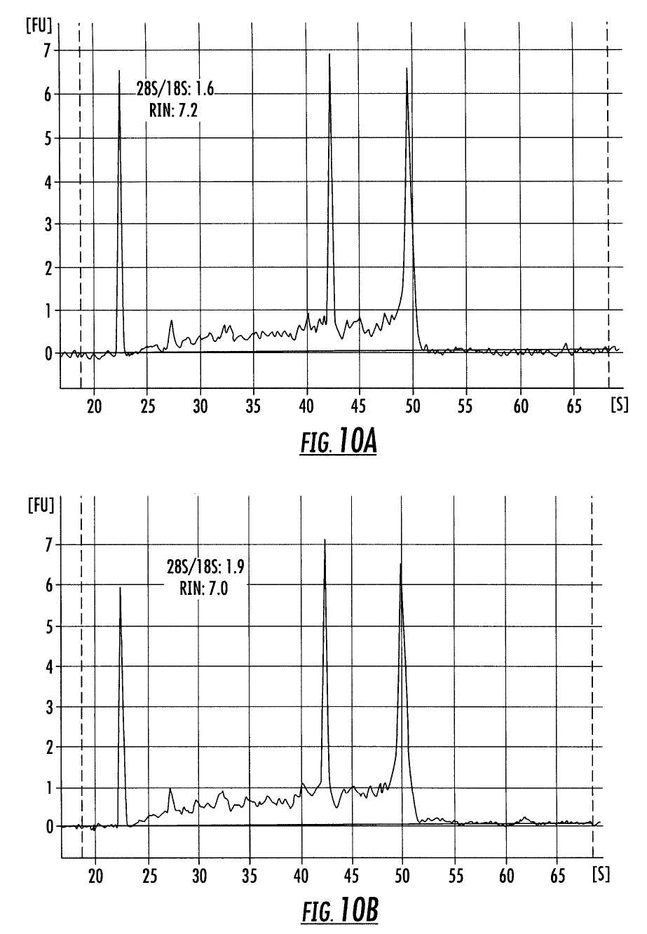

Agilent Bioanalyzer traces of the 30% tetrahydrofuran sample showed good quality of RNA as seen by a 28S/18S ratio of 2.2 and a RIN ratio of 9.7 (see FIG. 8B) and similar peaks when compared to the 35% ethanol sample (see FIG. 8A). QRT-PCR experiments that amplified GAPDH and B2M showed similar Cts when comparing the tetrahydrofuran and ethanol samples and overlapping amplification curves, suggesting that all the RNA samples had an equal quality (see FIG. 9).

RNA was also isolated from white blood cells using the Absolutely RNA® Miniprep Kit protocol described in Example 1 with the exception that tetrahydrofuran was used as a solvent in the binding buffer instead of ethanol. Tetrahydrofuran was added to the binding buffer at a final concentration of 10% to 40% to determine the effect of using different concentrations of tetrahydrofuran on the yield and purity of the RNA. Ethanol was added to the binding buffer at a final concentration of 35% as a control. As can be seen from Table 13, the highest yield of RNA was found at a final tetrahydrofuran concentration of 30% to 40%.

TABLE 13
RNA Yield Using Tetrahydrofuran Or Ethanol In Binding Mixture

| Tetrahydrofuran | Ethanol | RNA | |
|---|---|---|---|
| (%) | (%) | (ng/ul) | A260/280 |
| — | 35 | 36 | 2.02 |
| 10 | — | 16 | 1.9 |
| 20 | — | 20 | 2.08 |
| 30 | — | 35 | 2.05 |
| 40 | — | 37.6 | 2.03 |

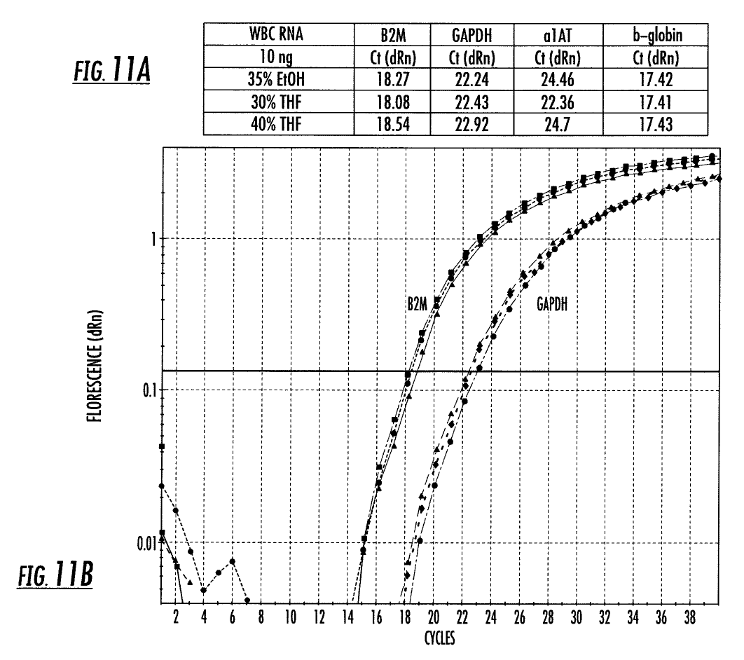

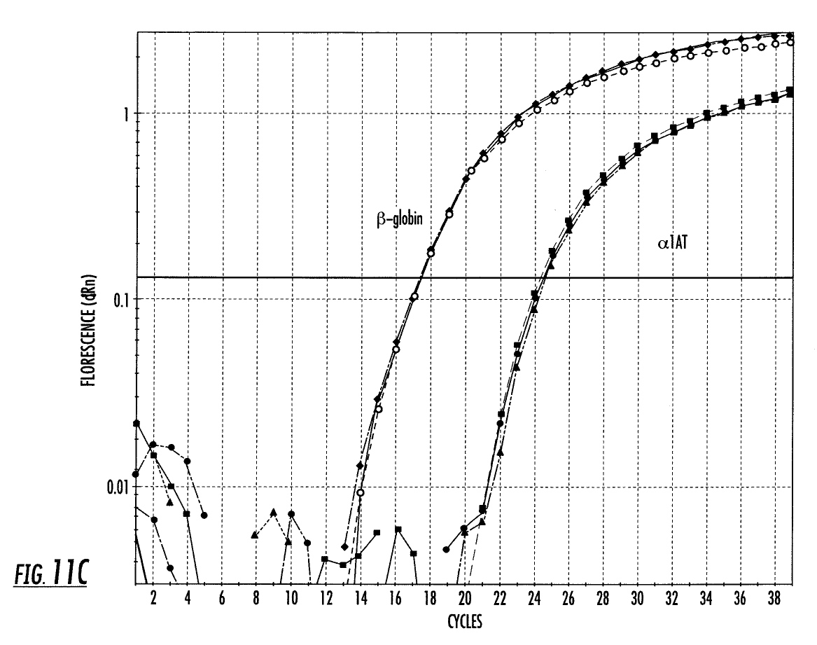

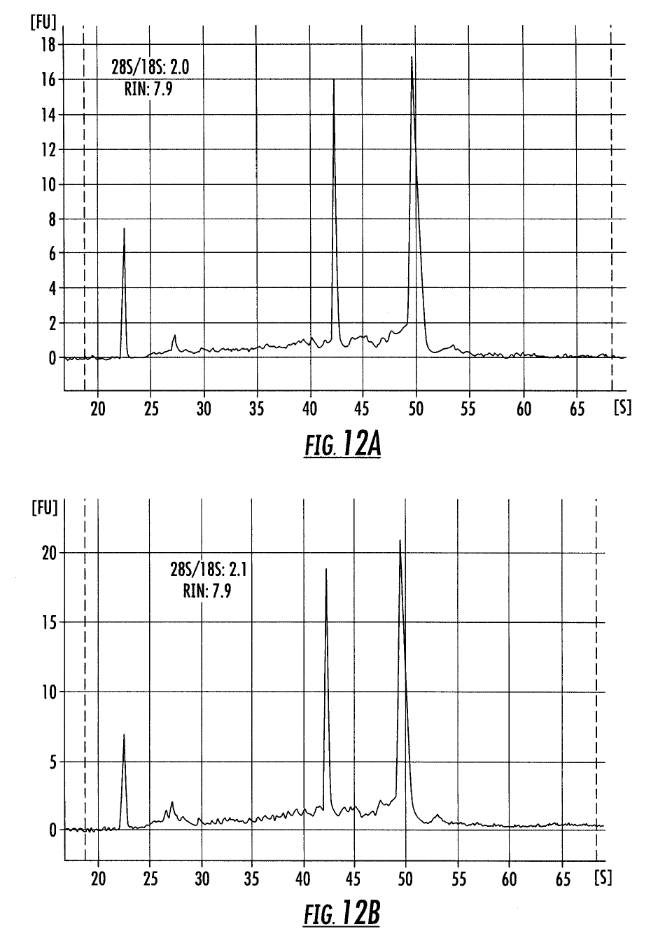

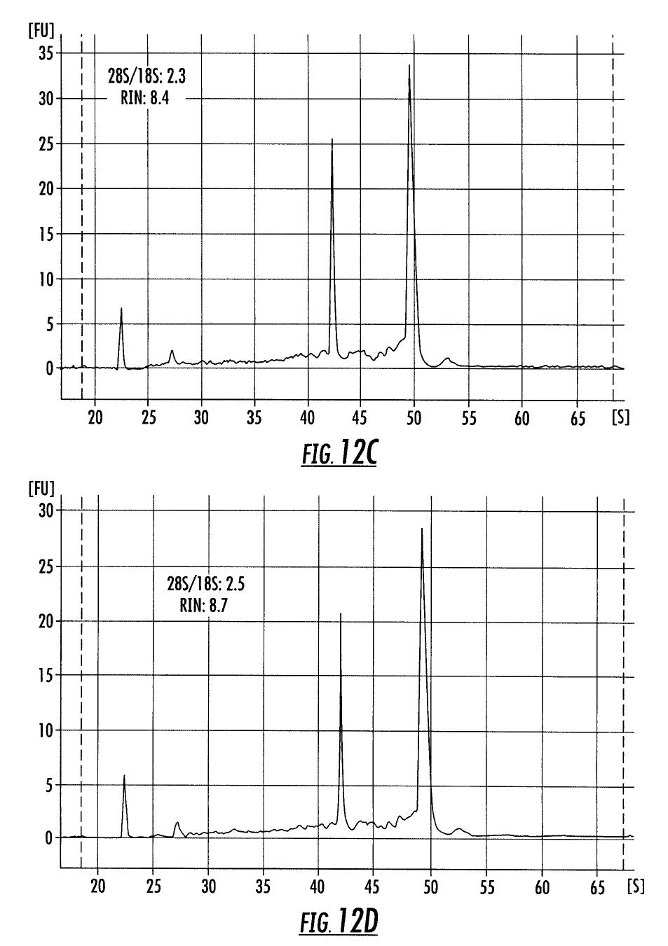

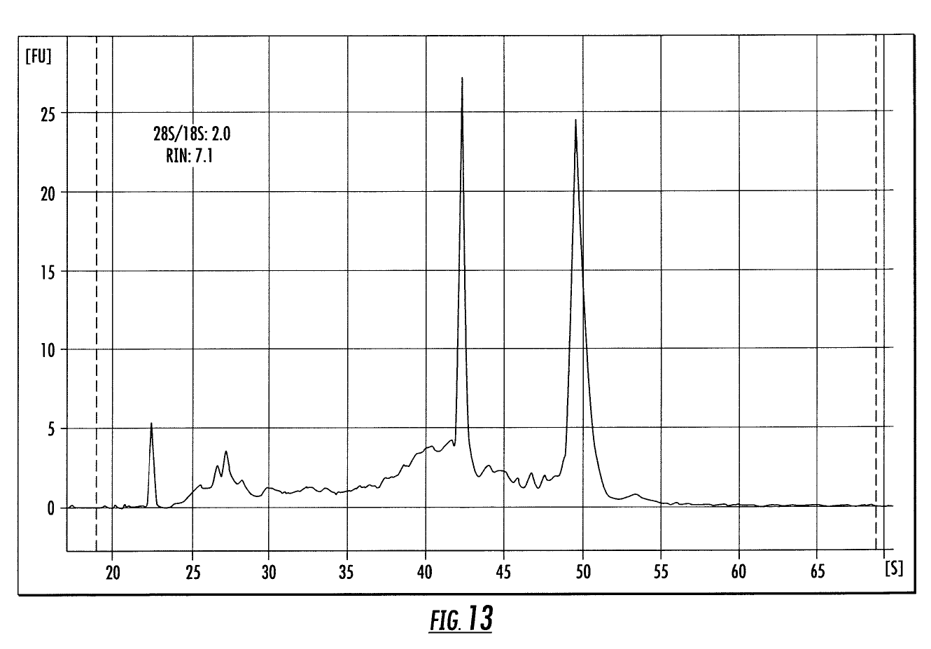

Agilent Bioanalyzer traces of the 30% tetrahydrofuran sample showed good quality of RNA as seen by a 28S/18S ratio of 1.9 and a RIN ratio of 7.0 (see FIG. 10B) and similar peaks when compared to the 35% ethanol sample (see FIG. 10A). QRT-PCR experiments that amplified GAPDH, B2M, beta-globin, and alpha-1AT showed similar Cts between the tetrahydrofuran and ethanol samples (see FIG. 11A) and overlapping amplification curves (see FIGS. 11B and 11C), suggesting that the RNA samples had an equal quality.

### Example 6

### Sulfolane as an Organic Solvent in Nucleic Acid Purification

RNA was isolated from a Jurkat cell line using the same protocol as described in Example 1 with the exception that sulfolane was used as a solvent in the binding buffer instead of ethanol. As seen in Table 14, sulfolane concentrations of 40% and 45% resulted in equivalent RNA yield and purity when compared to an ethanol concentration of 35%.

TABLE 14
RNA Yield Using Sulfolane Or Ethanol In Binding Mixture

| Sulfolane | | | |
|---|---|---|---|
| (%) | Ethanol (%) | RNA (ng/ul) | A260/280 |
| — | 35 | 65.81 | 2.1 |
| — | 35 | 67.3 | 2.09 |
| 10 | — | 3.72 | 1.82 |
| 15 | — | 4.55 | 2.13 |
| 20 | — | 4.63 | 1.77 |
| 25 | — | 7.42 | 1.94 |
| 30 | — | 27.81 | 2.02 |
| 35 | — | 63.8 | 2.09 |
| 40 | — | 67.79 | 2.1 |
| 45 | — | 70.49 | 2.08 |

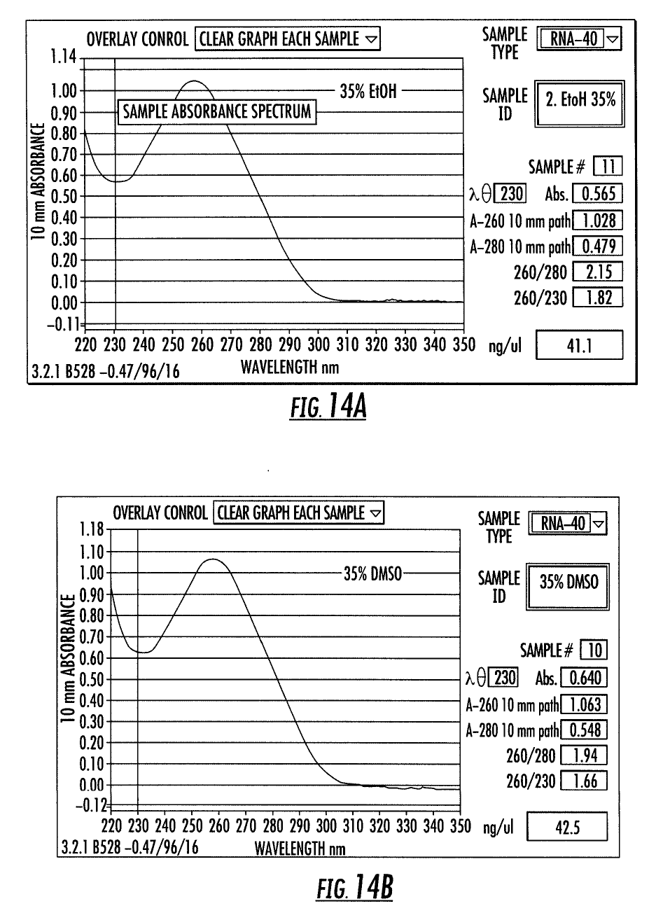

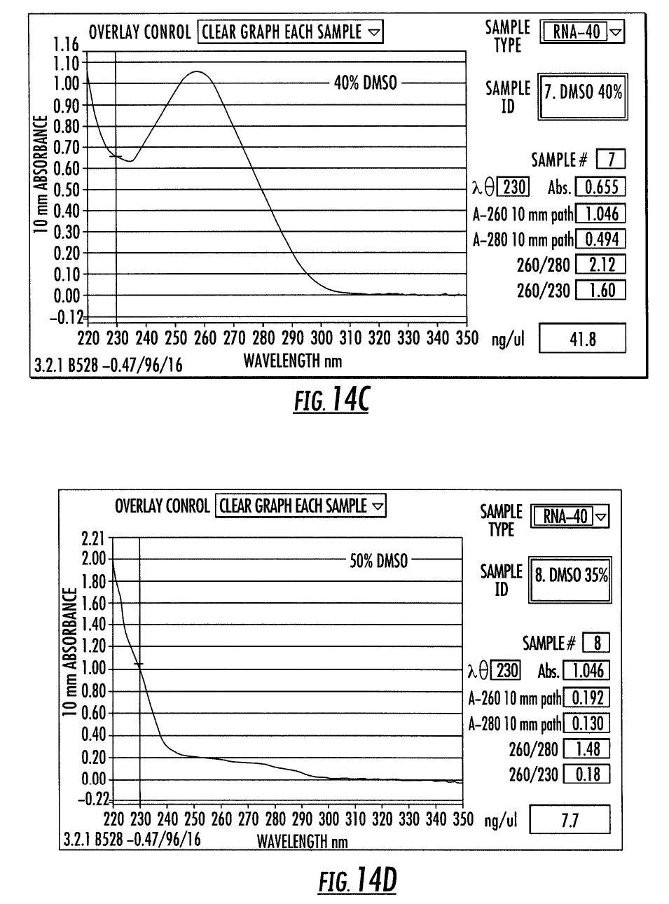

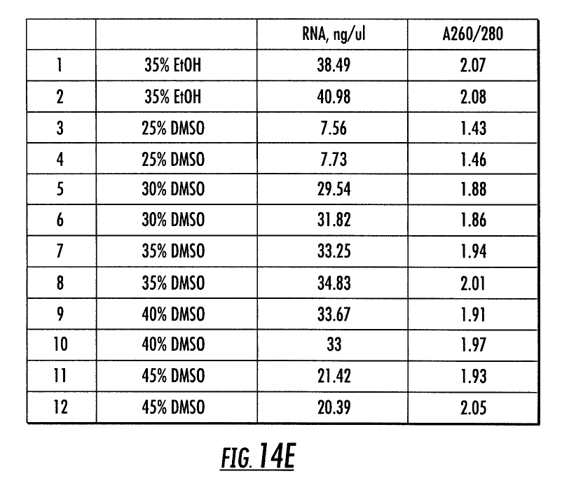

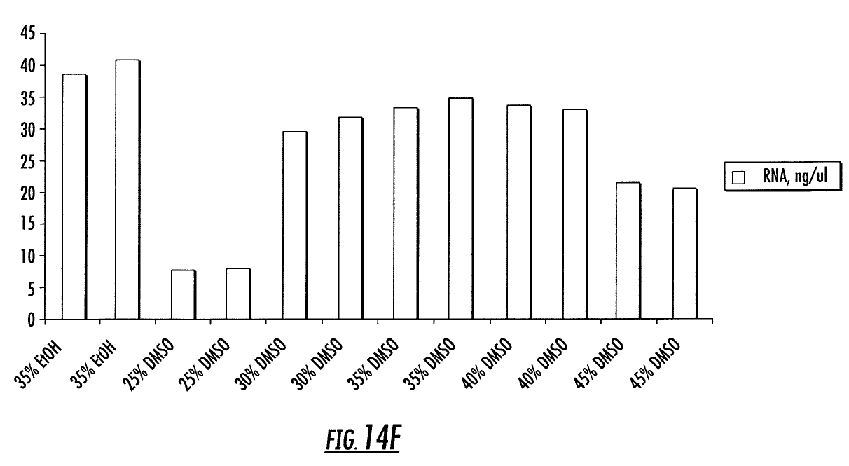

Agilent Bioanalyzer traces (see FIG. 12) demonstrated that 35% ethanol compares favorably with 45% sulfolane with respect to RIN number and 28/18 S ribosomal RNA ratios. More specifically, as shown in FIG. 12A, RNA isolated using 35% ethanol had a 28S/18S ratio of 2.0 and an RNA Integrity Number (RIN) of 7.9. In comparison, FIGS. 12B, 12C, and 12D show that RNA isolated using 45%, 40%, and 35% sulfolane in the RNA binding buffer, respectively, has an even higher 28S/18S ratio (2.1, 2.3, and 2.5, correspondingly) and higher RIN (7.9, 8.4, and 8.7, correspondingly). In summary, RIN number and 28/18 S ribosomal RNA ratio in the sulfolane series increase in the following order: 45% sulfolane (RIN=7.9; 28/18 S=2.0); 40% sulfolane (RIN=8.4; 28/18 S=2.3); and 35% sulfolane (RIN=8.7; 28/18 S=2.5). Overall, RNA isolated using either ethanol or sulfolane had a very good quality.

### Example 7

### 1,3-Dioxolane as an Organic Solvent in Nucleic Acid Purification

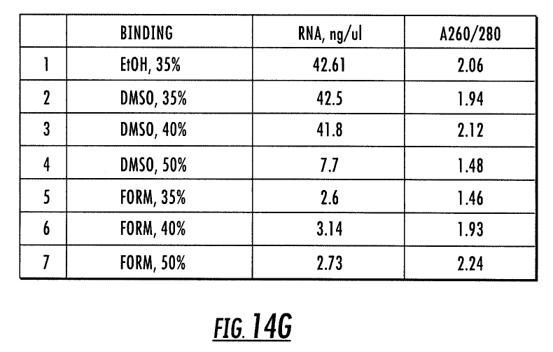

RNA was isolated from a Jurkat cell line using the same Absolutely RNA® Miniprep Kit protocol as described in Example 1 with the exception that 1,3-dioxolane was used as a solvent in the binding buffer instead of ethanol. As can be seen from FIG. 13, 1,3-dioxolane in the binding buffer resulted in a 28S/18S ratio of 2.0 and a RIN number of 7.1. These results are very similar to those obtained with ethanol.

### Example 8

### Comparison of DMSO and Formamide as an Organic Solvent in Nucleic Acid Purification

RNA was isolated from a Jurkat cell line using the same Absolutely RNA® Miniprep Kit protocol as described in Example 1 with the exception that dimethylsulfoxide (DMSO) or formamide was used as a solvent in the binding buffer instead of ethanol. DMSO was chosen in part because it has a dielectric constant of 38, which is below 80 and in the range of the majority of solvents that work well. Formamide was chosen for this experiment because its dielectric constant is 111, greater than the water dielectric constant of 80 and well outside the range of dielectric constants of solvents known to be useful in purification of RNA in this protocol.

As shown in FIG. 14, UV spectrophotometry demonstrated that 35% and 40% DMSO in the binding buffer performs equivalently to 35% ethanol (v/v) (Panels A, B, and C). DMSO at 50% (v/v) resulted in poor RNA yield (Panel D). Differing concentrations of DMSO were compared to 35% ethanol for the ability to influence purification of RNA in the protocol, and the results are depicted in FIGS. 14E and 14F. Panel E shows the results for 25%-40% (v/v) DMSO in tabular form, while Panel F shows the data in bar graph form. Thirty-five percent DMSO provided the highest yield of all DMSO concentrations tested, and showed equivalent purity to 35% ethanol.

In contrast to DMSO and other solvents discussed in the examples, formamide does not perform as well as ethanol in the purification protocol. As seen in FIG. 14, Panel G, while formamide can be used to obtain some nucleic acid, the quantity of material isolated is an order of magnitude lower than the amounts obtainable using other solvents. Similar results were obtained using trichloroacetic acid (TCA) (data not shown).

It will be apparent to those skilled in the art that various modifications and variations can be made in the practice of the present invention without departing from the scope or spirit of the invention. Other embodiments of the invention will be apparent to those skilled in the art from consideration of the specification and practice of the invention. It is intended that the specification and examples be considered as exemplary only, with a true scope and spirit of the invention being indicated by the following claims.

1. A method for the identification of an organic solvent useful in nucleic acid purification, said method comprising:
identifying an organic solvent with the following characteristics:
miscible, very soluble, or soluble in water;
a dielectric constant less than 80.

2. The method of claim 1, further comprising testing the organic solvent in a nucleic acid purification protocol.

3. The method of claim 1, wherein the organic solvent is a ketone, a nitrile, ether, sulfoxide, thiophene, alkane, sulfide, organic acid, phosphate, anhydride, ester, amide, amine (aliphatic, cyclic, or heterocyclic), or heterocyclic solvent containing one or more of the same or different heteroatoms.

4. The method of claim 3, wherein the thiophene is thiophene 1,1-dioxide.

5. The method of claim 1, wherein said nucleic acid purification protocol is used to purify RNA.

6. The method of claim 1, wherein the organic solvent is miscible in water.

7. The method of claim 1, wherein the organic solvent is very soluble or soluble in water, and wherein the method further comprises:
selecting an organic solvent having a solubility in water of 30%-80% (vol/vol); and
testing the selected organic solvent in a nucleic acid purification protocol.

8. The method of claim 7, further comprising:
selecting a solvent that is ketone, a nitrile, an ether, a sulfoxide, thiophene 1,1-dioxide, alkane, sulfide, organic acid, phosphate, anhydride, ester, amide, amine (aliphatic, cyclic, or heterocyclic), or heterocyclic solvent containing one or more of the same or different heteroatoms.

9. A composition for purification of a nucleic acid, said composition comprising:
an organic solvent identified by the method of claim 1, and water;
wherein the solvent is not ethanol, isopropanol, butanol, acetonitrile, tetrahydrofuran, or acetonitrile.

10. The composition of claim 9, further comprising one or more salts.

11. A kit comprising at least one container containing the composition of claim 8.

12. The kit of claim 11, further comprising some or all of the supplies and reagents used in a nucleic acid purification protocol.
